(19) United States
(12) Patent Application Publication
(10) Pub. No.: US 2022/0387317 A1
(43) Pub. Date: Dec. 8, 2022

(54) METHODS AND COMPOSITIONS RELATING TO SELECTIVE INTRACELLULAR DELIVERY OF CD38 INHIBITORS

(71) Applicant: Ohio State Innovation Foundation, Columbus, OH (US)

(72) Inventors: Sylvester Michael BLACK, New Albany, OH (US); Bryan Alan WHITSON, Westerville, OH (US); Brenda Faye CUSON, Blacklick, OH (US)

(21) Appl. No.: 17/775,236

(22) PCT Filed: Nov. 6, 2020
(86) PCT No.: PCT/US2020/059343

§ 371 Date: May 6, 2022

Related U.S. Application Data
(60) Provisional application No. 62/932,152, filed on Nov. 7, 2019.

(51) Int. Cl. A61K 9/127; A61K 31/352; A61K 31/4709; A61K 31/7048

(52) U.S. Cl. CPC A61K 9/127; A61K 31/352; A61K 31/4709; A61K 31/7048; B82Y 5/00

(57) ABSTRACT

Disclosed are compositions engineered nanovesicles and compositions comprising CD38 inhibitors and methods of using said nanovesicles and compositions to treat an inflammatory liver disease as well as in inhibiting, reducing, or repairing tissue damage to a donor organ or tissue during a transplantation procedure. In one aspect, also disclosed are method of preparing a donor organ or tissue for transplant comprising contacting the organ or tissue with the engineered nanovesicle or compositions disclosed herein.

### I. CROSS-REFERENCE TO RELATED APPLICATIONS

This application claims the benefit of U.S. Provisional Application No. 62/932,152, filed on Nov. 7, 2019, which is incorporated herein by reference in its entirety.

### II. BACKGROUND

In the United States, ˜38,000 people die yearly of cirrhosis with transplantation as the only effective treatment for end-stage liver disease. While survival rates after liver transplantation are excellent, recipient demand far exceeds donor supply. In order to meet demand, the donor pool has expanded to include marginal organs from donors with advanced age or other medical co-morbidities. Currently, the post-transplantation function of a donor liver is predicted using imprecise laboratory tests such as hepatic transaminases, lactate clearance, and coagulation profiles. Marginal donor organs function sub-optimally when transplanted, thus they tend be discarded at a higher rate due to concern over subsequent risk to the recipient. Marginal organs are at greater risk during ischemia reperfusion injury (IRI), which is biphasic with a period of ischemia followed by reperfusion after restoration of blood flow.

IRI occurs due to the accumulation of reactive oxygen species (ROS) and pro-inflammatory mediators that further injure the graft. The high susceptibility of marginal organs to IRI can result in early allograft dysfunction or even primary non-function in the recipient. Since marginal donor organs have increased susceptibility to IRI they must be intrinsically different with yet unidentified factors contributing to enhanced injury. Predicting which marginal donor organs will tolerate transplantation and maintain function addresses a key gap in knowledge of how to safely expand the donor pool and allow for increased organ utilization. Moreover, new transplantation methodologies are needed that can avoid damage that results in marginal donor organs or can rescue marginal organs rendering them suitable for transplantation.

### III. SUMMARY

Disclosed are methods and compositions related to inhibiting CD38 for the treatment of inflammatory conditions and reducing damage to donor organs in transplantation.

In one aspect, disclosed herein are engineered nanovesicles comprising one or more macrophage targeting moieties (such as, for example an antibody specific for a cell surface biomarker specific to the macrophage in the interstitial compartment of a particular tissue or organ) and an inhibitor of CD38 (such as, for example, an RNAi, oligonucleotide, antibody, or small molecule). In one aspect, the small molecule inhibitor of CD38 can comprise a thiazoloquin(az)olin(on)e compound (such as, for example compound 78C), apigenin, kuromanin, or luteolinidin. In one aspect, the macrophage targeting moiety targets macrophage in the interstitial compartment (for example, by having specificity for a cell surface biomarker specific to the macrophage in the interstitial compartment of a particular tissue or organ). Also disclosed herein are pharmaceutical composition comprising the engineered nanovesicle of any preceding aspect.

In one aspect, disclosed herein are methods of treating, reducing, inhibiting, decreasing, ameliorating, and/or preventing an inflammatory disease (such as, for example, an inflammatory liver disease including, but not limited to Hyperlipidemia, fatty liver disease (steatosis), steatohepatitis, metabolic syndrome, Phenylketonuria (PKU), Maple syrup urine disease (MSUD), Gaucher's disease, hypercholesterolemia, hypertriglyceridemia, hyperthyroidism, hypothyroidism, dyslipidemia, hypolipidemia, and galactosemia, alcoholic liver disease (ALD), or non-alcoholic fatty liver disease (such as, for example, non-alcoholic fatty liver (NAFL) or non-alcoholic steatohepatitis (NASH)), liver fibrosis, lung inflammatory disease (such as, for example, acute lung injury, acute respiratory distress syndrome (ARDS), transfusion induced acute lung injury (TRALI), or ventilator induced lung injury), acute inflammation, and/or sepsis comprising administering to a subject with an inflammatory liver disease the engineered nanovesicle or the pharmaceutical composition of any preceding aspect. For example, disclosed herein are methods of treating, inhibiting, reducing, decreasing, ameliorating, and/or preventing an inflammatory liver disease comprising administering to a subject with an inflammatory liver disease an inhibitor of CD38 (such as, for example, an RNAi, oligonucleotide, antibody, or small molecule). In one aspect, the small molecule inhibitor of CD38 can comprise a thiazoloquin(az)olin(on)e compound (such as, for example compound 78C), apigenin, kuromanin, or luteolinidin.

Also disclosed herein are methods of preparing a donor organ (such as, for example liver, lung, heart, kidney, trachea, or pancreas) or tissue (bones, skin, tendons, cornea, vascular tissue, or heart valves) for transplant comprising contacting the organ or tissue with the engineered nanovesicle or the pharmaceutical composition of any preceding aspect

In one aspect, disclosed herein are methods of inhibiting, reducing, or repairing tissue damage to a donor (such as, for example liver, lung, heart, kidney, trachea, or pancreas) or tissue (bones, skin, tendons, cornea, vascular tissue, or heart valves) during a transplantation procedure comprising contacting the organ or tissue with the engineered nanovesicle with the engineered nanovesicle or the pharmaceutical composition of any preceding aspect.

In one aspect the engineered nanovesicle or pharmaceutical composition is delivered to a donor subject comprising the donor tissue or organ prior to removal of the organ or tissue. In some aspect, the nanovesicle or pharmaceutical composition is delivered to the organ or tissue via ex vivo organ perfusion, solution flush, and/or static storage solution such as for example a cold static storage solution or normothermic solution). In some aspects, the engineered nanovesicle or pharmaceutical composition can be administered prior to transplantation or as part of a post-transplantation procedure.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C and 1D show that pre-incubation of CD38 unconjugated Ab blocks binding of CD38 conjugated flow Ab on surface. Surface CD38 was labeled with an eFluor660-CD38 Ab following pre-incubation of surface CD38 with non-labeled CD38 Ab or isotype IgG isotype

FIGS. 2A, 2B, 2C and 2D show cellular location of CD38 by flow-cytometry. For intracellular CD38 staining, cells were pre-incubated with CD38Ab (not labeled) then incubated with an eFluor660-CD38 Ab. Data show it is compared with surface CD38-ef660 only.

FIGS. 3A, 3B, 3C and 3D show ImageStream quantification of CD38 localization. Cells were blocked and stained for surface CD38 or intracellular CD38 then analyzed by ImageStream by applying cell specific masks. DAPI was used as a nuclear marker and Albumin, GFAP, CD31 and Mac-Subset were used for hepatocyte, HSC, LSEC and KC markers, respectively.

FIG. 4 shows histological analysis of liver steatosis and fibrosis. H&E 10×; Trichrome 20×.

FIGS. 5A, 5B, 5C, and 5D show MCD diet upregulates genes related to fibrosis (5A) and proinflammatory cytokines (5B) in liver tissue and increases proinflammatory protein in plasma. Liver tissue from MCD diet mice had (A) increased MMP-9, (B) TGF-β, TNF-α, and CCL-2 gene expression in liver tissue; (5C and 5D) IL-6 and TNF-α was also increased in the plasma of MCD diet mice.

FIGS. 6A, 6B, 6C, and 6D show MCD diet induced alterations in NAD and ATP levels in liver tissue. FIG. 6A shows NAD was decreased in fatty livers (6B) while NADH showed no difference; however (6C) NADH/NAD ratios and (6D) ATP showed significant decreases in fatty livers.

FIGS. 7A, 7B, 7C, 7D, and 7E show MCD diet causes increased CD38 protein expression and activity and decreased Sirt family protein and gene expression in liver tissue. FIG. 7A shows western blot showed increased CD38 and decreased Sirt-1 protein expression in MCD diet livers; (7B) while qPCR showed no significant difference in CD38 expression, (7C) CD38 and (7D) cyclase activities were increased in MCD diet livers: (7E) gene expression of Sirt-1, 4, 5, and 6 were decreased by MCD diet.

FIGS. 8A, 8B, 8C, and 8D show CD38 expression and location in major liver cell types. Liver cell types were isolated from mice fed normal or MCD diet. MCD diet did not induce significant changes in CD38 expression in (8A) liver sinusoidal endothelial cells (LSECs) or (8B) Kupffer cells; however, (8C) surface CD38 expression was decreased in hepatic stellate cells (HSCs) and (8D) increased in hepatocytes.

FIG. 9 shows CD38 expression increases in hepatocytes as shown by immunohistochemistry. 20×

FIGS. 10A, 10B, 10C, and 10D show flow cytometric (10A, 10B, and 10C) and ImageStream analysis (10D) of CD38 expression in hepatocytes and small hepatocytes. FIGS. 10A and 10B show small hepatocytes increased by 10-fold in fatty livers; (10C) CD38 expression was found to be increased in these small hepatocytes compared to regular hepatocytes; (10D) ImageStream analysis confirmed the increase of surface CD38 expression in small hepatocytes.

FIG. 11 shows hypoxia (1.1% O2) increased CD38 expression and activity on HSCs. Western blot of HSC and KC CD38 expression during normoxia and hypoxia. CD38 has increased expression during hypoxia to a great extent in HSC compared to KC; n=3.

FIG. 12 shows CD38 activity assay demonstrates that CD38 is similarly increased during hypoxia to a greater extent in HSCs compared with KCs; n=3. Data is shown as mean±SD.

FIGS. 13A, 13B, and 13C show CD38 inhibition with Kuromanin in primary rat HSCs. Exposed to 3 h of hypoxia followed by oxidant stress (H₂O₂) in the presence of increasing amounts of Kuromanin (1-20 μM) show 13A) increased viability and 13B) decreased intracellular ROS formation, and 13C) decreased ET-1 release (at 20 μM Kuromanin). n=3/group. Data is shown as mean±SD.

FIG. 14 shows CD38 activity during IRI decreases with Inhibitor treatment.

FIGS. 15A, 15B, and 15C show CD38 expression, activity and hepatocellular injury increase with duration of ischemia in a rat hilar clamp model of IRI. FIG. 15A shows CD38 expression, 15B) ALT, and 15C) CD38 enzymatic activity increase with a longer ischemic time, representing a greater reperfusion injury. n=3 per group Data is shown as mean±SD.

FIG. 16 shows intrahepatic delivery of CD38 inhibitor attenuates liver IRI. Intrahepatic delivery of the CD38 inhibitor Apigenin decreased injury during a rat liver hilar clamp model of IRI, 1 h of ischemia and 6 h of reperfusion.

FIGS. 17A, 17B, 17C, and 17D show that CD38 is present only on HSCs and some KCs as indicated by 17A) flow cytometry and 17B) fluorescent microscopy. FIG. 17C shows western blot show that CD38 expression is increased in HSCs during hypoxia compared to KC and 17D) fluorescent activity assay shows CD38 activity is greater in HSCs compared with KCs

FIGS. 18A, 18B, 18C, 18D, 18E, and 18F shows that rats fed a methionine choline deficient (MCD) diets have livers with similar inflammation to those of a “high risk” human donor organ: FIG. 18A shows control rats have very little steatosis by H&E and 18B shows low CD38 expression by IHC. FIG. 18C shows MCD rats show severe steatosis by H&E and 18D shows high expression of CD38 by IHC. FIG. 18E shows discarded human donor liver has severe steatosis by H&E and 18F shows moderate CD38 expression by IHC.

FIGS. 19A, 19B, 19C, 19D, 19E, and 19F shows the effects of 78C (FIGS. 19B, 19C and 19F) or Luteolinidin (FIG. 19A, 19D, or 19E) on the viability of rat hepatocytes (19A and 19B), rat hepatic stellate cells (19C), rat sinusoidal endothelial cells (19D), or Kupffer cells (19E and 19F).

FIG. 20 shows the effects of pretreatment and co-treatment on viability of hepatocytes.

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

The term “therapeutically effective” refers to the amount of the composition used is of sufficient quantity to ameliorate one or more causes or symptoms of a disease or disorder. Such amelioration only requires a reduction or alteration, not necessarily elimination.

“Therapeutically effective amount” or “therapeutically effective dose” of a composition (e.g. a composition comprising an agent) refers to an amount that is effective to achieve a desired therapeutic result. In some embodiments, a desired therapeutic result is the control of diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. INFLAMMATORY DISEASE AND TRANSPLANTATION

Liver transplantation is the only effective treatment for cirrhosis. With advancements in surgical technique, organ preservation, anesthesia, critical care, and immunosuppression, patients undergoing liver transplantation have 1-year survival rates of 85-95% and a median survival of 11.6 years compared to 3.1 years without a transplant 2 Since there are ˜15,000 patients on the waiting list with only ˜7,000 operations performed annually, the yearly wait-list mortality for liver transplantation can exceed 30% in some areas of the U.S. Marginal organs also have increased cost, resource utilization, and complication rates and are more likely to be discarded when compared to “standard” criteria donor organs. The number and diminished quality of available donor organs are limiting factors to a more widespread application of liver transplantation and have led to decreased donor organ utilization, lower quality grafts, and poorer outcomes. While the demand for donor livers is high, socio-cultural and psychological barriers to donation and decreases in traumatic deaths of younger, healthier donors has made marginal organs a vital and viable means to increase the donor pool.

After transplantation, marginal organs tend to have decreased viability and/or function with higher recipient morbidly and mortality. The most common chronic liver disease, non-alcoholic fatty liver disease (NAFLD), affects 30% of those in western countries and 70-80% of obese individuals, thus making steatotic livers the most common type of marginal organ used to extend the donor pool. While few studies of severely steatotic grafts (macrosteatosis of >60%) exist, there are reports of extremely high rates of primary non-function and recipient mortality in excess of 75%. While using grafts with mild macrosteatosis (<30%) generally have good clinical outcomes, moderately steatotic grafts (30-60%) must be used with caution. These grafts tend to be more susceptible to severe injury and dysfunction when compared with typical donor organs. Therefore, rigorous scientific research is critical to better identify and match these organs to recipients.

Within the hepatic sinusoid are perivascular hepatic stellate cells (HSC) that, when injured or activated, are a potent source of pro-inflammatory mediators and ROS that can contribute to graft dysfunction.

The hepatic stellate cell (HSC) is well-studied and characterized for its role in chronic inflammatory conditions; however, it's role in acute injury or the response to ischemia reperfusion injury (IRI) occurring with transplantation is not as well-characterized. HSCs make up 5-8% of liver cells, and due to their perivascular location within the hepatic sinusoid, they have increased potential to exacerbate the injury that occurs with the donation process. Once injured, HSCs produce a number of pro-inflammatory mediators (ET-1, IL-1, TNF, IL-6) and reactive oxygen species (ROS) that can greatly impact donor organ function. The inflammatory ectoenzyme CD38, which is primarily constitutively expressed by HSCs and some Kupffer cells (KC) in the liver, has been shown to have enhanced expression in relation to the activation status of HSCs from patients with chronic liver disease. Furthermore, during IRI, hypoxia, and inflammation, CD38 has been shown to be activated by ROS through NAD(P)H Oxidase (NOX) resulting in increased intracellular Ca2+ through secondary Ca2+ messengers cyclic ADP-ribose (cADPR) and nicotinic acid adenine dinucleotide phosphate (NAADP). Elevated intracellular Ca2+ is especially important in the pathophysiology of the HSC as activated HSCs are contractile secondary to expression of a-smooth muscle actin (a-SMA) and other motor proteins and also express pro-inflammatory cytokines (IL-6) and intercellular adhesion molecules (ICAM and VCAM). Therefore, CD38 can act as a regulator of HSC activation and effector function. Additionally, many marginal organs have populations of prior activated, hyperinflammatory HSCs, and, with ROS as important mediators in the activation of CD38, this can help to explain the microcirculatory (hepatic sinusoid) dysfunction and enhanced hepatic injury seen with these organs after transplantation. Stated slightly differently, the activated CD38 explains why marginal organ HSCs, already skewed toward an inflammatory phenotype respond disproportionately to IRI compared to HSCs of a normal organ resulting in greater graft injury. The intrinsic inflammatory state of marginal donor organs with activated HSCs and high CD38 expression/activity lead to enhanced injury during IRI critically contributing to the decreased function of these grafts after transplantation.

Because increased CD38 expression/activity can be associated with enhanced injury during IRI, it is understood and herein contemplated that inhibiting or reducing CD38 expression/activity can inhibit, reduce, decrease, ameliorate, and/or prevent tissue or organ damage during an ischemia, reperfusion, and/or transplantation. Thus, in one aspect, disclosed herein are methods of preparing a donor (such as, for example liver, lung, heart, kidney, trachea, or pancreas) or tissue (bones, skin, tendons, cornea, vascular tissue, or heart valves) for transplantation comprising contacting the organ or tissue with any of the CD38 inhibitor comprising engineered nanovesicle or pharmaceutical compositions disclosed herein. For example, disclosed herein are methods of preparing a donor organ or tissue for transplantation comprising contacting the organ or tissue with an engineered nanoparticle comprising one or more CD38 inhibitors (such as, for example, an RNAi, oligonucleotide, antibody, or small molecule) and one or more targeting moieties (such as, for example, an antibody that targets macrophage contained in the interstitial compartment of a donor organ or tissue including, but not limited to an antibody that targets CD14, CD16, CX3CR1, SiglecF, CD206, F4/80, CD64, CD80, CD86, MHC II, CD68, CD31/PECAM-1, P-selectin, E-selectin, CD54/Intercellular Adhesion Molecule 1 (ICAM-1), CD106/vascular cell adhesion molecule 1 (VCAM-1), and/or CCR2). In one aspect, the small molecule inhibitor of CD38 can comprise a thiazoloquin(az)olin(on)e compound (such as, for example compound 78C), apigenin, kuromanin, or luteolinidin.

It is understood and herein contemplated that the organ or tissue can be contacted with an engineered nanoparticle comprising one or more CD38 inhibitors (such as, for example, an RNAi, oligonucleotide, antibody, or small molecule) and one or more targeting moieties (such as, for example, an antibody that targets macrophage contained in the interstitial compartment of a donor organ or tissue including, but not limited to an antibody that targets CD14, CD16, CX3CR1, SiglecF, CD206, F4/80, CD64, CD80, CD86, MHC II, CD68, CD31/PECAM-1, P-selectin, E-selectin, CD54/Intercellular Adhesion Molecule 1 (ICAM-1), CD106/vascular cell adhesion molecule 1 (VCAM-1), and/or CCR2) ex vivo for any amount of time sufficient to have an efficacious outcome. In one aspect, the organ or tissue can be contacted with an engineered nanoparticle ex vivo for 1, 2, 3, 4, 5 6, 7, 8, 9, 10, 15, 20, 25, 30, 35, 40, 45, 50, 55, 60, 70, 80, 90, 100, 110, 120, 150, 180 minutes, 4, 5, 6, 7, 8, 9, 10, 11, 12, 13, 14, 15, 16, 17, 18, 19, 20, 21, 22, 23, 24, 30, 36, 42, 48, 60, 72 hours, 4, 5, 6, or 7 days.

It is understood and herein contemplated that methodologies that work for preparing a donor tissue or organ for transplantation and avoid CD38 mediated injuries that would otherwise leave the tissue or organ marginal or unsuitable for use as a donor organ can be applied to rescue an otherwise marginal or unsuitable organ from injury and restore said tissue or organ to a suitable state. Thus, in one aspect, disclosed herein are methods of inhibiting, reducing, or repairing tissue damage to a donor (such as, for example liver, lung, heart, kidney, trachea, or pancreas) or tissue (bones, skin, tendons, cornea, vascular tissue, or heart valves) during a transplantation procedure comprising contacting the organ or tissue with any of the CD38 inhibitor comprising engineered nanovesicle or pharmaceutical compositions disclosed herein. For example, disclosed herein are methods of inhibiting, reducing, or repairing tissue damage to a donor organ or tissue during a transplantation procedure comprising contacting the organ or tissue with an engineered nanoparticle comprising one or more CD38 inhibitors (such as, for example, an RNAi, oligonucleotide, antibody, or small molecule) and one or more targeting moieties (such as, for example, an antibody that targets macrophage contained in the interstitial compartment of a donor organ or tissue including, but not limited to an antibody that targets CD14, CD16, CX3CR1, SiglecF, CD206, F4/80, CD64, CD80, CD86, MHC II, CD68, CD31/PECAM-1, P-selectin, E-selectin, CD54/Intercellular Adhesion Molecule 1 (ICAM-1), CD106/vascular cell adhesion molecule 1 (VCAM-1), and/or CCR2). In one aspect, the small molecule inhibitor of CD38 can comprise a thiazoloquin(az)olin(on)e compound (such as, for example compound 78C), apigenin, kuromanin, or luteolinidin.

Due to the significant role of CD38 in damage associated with tissue or organ transplantation, it is contemplated that the disclosed methodologies could further be applied for the treatment of any inflammatory condition mediated increased CD38 expression and/or activation and displaying increased inflammation due to increases in CD38. As used herein, “Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

In one aspect, disclosed herein are methods of treating, reducing, inhibiting, decreasing, ameliorating, and/or preventing an inflammatory disease (such as, for example, an inflammatory liver disease) comprising administering to a subject with an inflammatory disease any of the CD38 inhibitor comprising engineered nanovesicle or pharmaceutical compositions disclosed herein (including, therapeutically effective amounts of said engineered nanovesicle or pharmaceutical compositions). For example, disclosed herein are methods of reducing, inhibiting, decreasing, ameliorating, and/or preventing an inflammatory liver disease comprising administering to a subject with an inflammatory liver disease an inhibitor of CD38 (such as, for example, an RNAi, oligonucleotide, antibody, or small molecule). In one aspect, the small molecule inhibitor of CD38 can comprise a thiazoloquin(az)olin(on)e compound (such as, for example compound 78C), apigenin, kuromanin, or luteolinidin. The CD38 inhibitor can be delivered in a pharmaceutical composition with or without the presence of any of the engineered nanoparticles disclosed herein or simply that nanoparticles in a suitable delivery vehicle. In one aspect, disclosed herein are methods of reducing, inhibiting, decreasing, ameliorating, and/or preventing an inflammatory liver disease comprising administering to a subject with an inflammatory liver disease an engineered nanoparticle comprising one or more CD38 inhibitors (such as, for example, an RNAi, oligonucleotide, antibody, or small molecule) and one or more targeting moieties (such as, for example, an antibody that targets macrophage contained in the interstitial compartment of a donor organ or tissue including, but not limited to an antibody that targets CD14, CD16, CX3CR1, SiglecF, CD206, F4/80, CD64, CD80, CD86, MHC 11, CD68, CD31/PECAM-1, P-selectin, E-selectin, CD54/Intercellular Adhesion Molecule 1 (ICAM-1), CD106/vascular cell adhesion molecule 1 (VCAM-1), and/or CCR2). In one aspect, the small molecule inhibitor of CD38 can comprise a thiazoloquin(az)olin(on)e compound (such as, for example compound 78C), apigenin, kuromanin, or luteolinidin.

It is understood and herein contemplated that the inflammatory disease mediated by CD38 can comprise any inflammatory disease known in the art including but not limited to inflammatory liver diseases such as, for example, Hyperlipidemia, fatty liver disease (steatosis), steatohepatitis, metabolic syndrome, Phenylketonuria (PKU), Maple syrup urine disease (MSUD), Gaucher's disease, hypercholesterolemia, hypertriglyceridemia, hyperthyroidism, hypothyroidism, dyslipidemia, hypolipidemia, and galactosemia, Alcoholic liver disease (ALD), or non-alcoholic fatty liver disease (such as, for example, non-alcoholic fatty liver (NAFL) or non-alcoholic steatohepatitis (NASH)), liver fibrosis, lung inflammatory disease (such as, for example, acute lung injury, acute respiratory distress syndrome (ARDS), transfusion induced acute lung injury (TRALI), or ventilator induced lung injury), sepsis autoimmune disease, autoinflammatory disease, and acute inflammation. Other inflammatory conditions that can be treated, inhibited, reduced, decreased, ameliorated and/or prevented by the disclosed methods include, but are not limited to autoinflammatory diseases and autoimmune diseases.

As used herein “autoinflammatory disorders refer to disorders where the innate immune response attacks host cells. Examples of autoimmune diseases that can be treated by any of the CD38 inhibitor comprising engineered nanovesicle or pharmaceutical compositions disclosed herein include, but are not limited to asthma, graft versus host disease, allergy, transplant rejection, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF), Tumor Necrosis Factor (TNF)-Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1ß (IL-1ß) Receptor Antagonist (DIRE) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behgets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome, Acute Febrile Neutrophilic Dermatosis, X-linked familial hemophagocytic lymphohistiocytosis, and Lyn kinase-associated Autoinflammatory Disease (LAID).

As used herein, “autoimmune disease” refers to a set of diseases, disorders, or conditions resulting from an adaptive immune response (T cell and/or B cell response) against the host organism. In such conditions, either by way of mutation or other underlying cause, the host T cells and/or B cells and/or antibodies are no longer able to distinguish host cells from non-self-antigens and attack host cells baring an antigen for which they are specific. Examples of autoimmune diseases that can be treated by any of the CD38 inhibitor comprising engineered nanovesicle or pharmaceutical compositions disclosed herein include, but are not limited to Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Balb disease, Behcet's disease, Benign mucosal emphigoid, Bickerstaffs encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (tBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)).

In one aspect the engineered nanovesicles or pharmaceutical compositions disclosed herein can be delivered to a donor subject comprising the donor tissue or organ prior to removal of the (such as, for example liver, lung, heart, kidney, trachea, or pancreas) or tissue (bones, skin, tendons, cornea, vascular tissue, or heart valves) or directly to the donor tissue or organ. In some aspect, the nanovesicle or pharmaceutical composition is delivered to the organ or tissue via ex vivo organ perfusion (EVOP) including, but not limited to normothermic ex-vivo liver perfusion (NEVLP), solution flush, and/or static storage solution such as for example a cold static storage solution or normothermic solution). In some aspects, the engineered nanovesicle or pharmaceutical composition can be administered prior to transplantation or as part of a post-transplantation procedure.

### C. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular engineered nanoparticle comprising one or more CD38 inhibitors and one or more macrophage targeting moieties is disclosed and discussed and a number of modifications that can be made to a number of molecules including the engineered nanoparticle comprising one or more CD38 inhibitors and one or more macrophage targeting moieties are discussed, specifically contemplated is each and every combination and permutation of engineered nanoparticle comprising one or more CD38 inhibitors and one or more macrophage targeting moieties and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

As noted above, the disclosed methods of treating, reducing, inhibiting, and/or preventing an inflammatory disease; methods of preparing a donor (such as, for example liver, lung, heart, kidney, trachea, or pancreas) or tissue (bones, skin, tendons, cornea, vascular tissue, or heart valves) for transplantation; and/or methods of inhibiting, reducing, or repairing tissue damage to a donor (such as, for example liver, lung, heart, kidney, trachea, or pancreas) or tissue (bones, skin, tendons, cornea, vascular tissue, or heart valves) during a transplantation procedure comprise the use of engineered nanoparticles comprising a CD38 inhibitor and a macrophage targeting moiety. Accordingly, in one aspect, disclosed herein are engineered nanovesicles comprising one or more macrophage targeting moieties (such as, for example an antibody specific for a cell surface biomarker specific to the macrophage in the interstitial compartment of a particular tissue or organ) and an inhibitor of CD38 (such as, for example, an RNAi, oligonucleotide, antibody, or small molecule). In one aspect, the small molecule inhibitor of CD38 can comprise a thiazoloquin(az)olin(on)e compound (such as, for example compound 78C), apigenin, kuromanin, or luteolinidin. In one aspect, the macrophage targeting moiety targets macrophage in the interstitial compartment (for example, by having specificity for a cell surface biomarker specific to the macrophage in the interstitial compartment of a particular tissue or organ). Also disclosed herein are pharmaceutical composition comprising the engineered nanovesicle of any preceding aspect.

The CD38 inhibitor used in the methods disclosed herein and the engineered nanoparticles can comprise any CD38 inhibitor known in the art including, but not limited to anti-CD38 antibodies, immunotoxins, RNAi, anti-sense oligonucleotides, and/or small molecules. In one aspect, the CD38 inhibitor comprises a bioflavonoid (such as, for example, kuromanin, apigenin, or luteolinidin) or a thiazoloquin(az)olin(on)e compound (such as, for example compound 78C). For example, in one aspect, disclosed herein are engineered nanovesicles comprising one or more macrophage targeting moieties (such as, for example an antibody specific for a cell surface biomarker specific to the macrophage in the interstitial compartment of a particular tissue or organ) and compound 78C. In one aspect compound 78C is administered at a concentration of less than 50 μM.

It is understood and herein contemplated that the disclosed engineered nanoparticle comprise one or more targeting moieties to deliver the CD38 inhibitor and any additional cargo to a target cell, organ, or tissue and avoid any pleiotropic effect from off-target inhibition of CD38. In one aspect, it is understood and herein contemplated that the one or more targeting moieties can specifically target macrophage contained in the interstitial compartment of a donor organ or tissue as well as endothelium. In one aspect, the one or more targeting moieties include, but are not limited to CD14, CD16, CX3CR1, SiglecF, CD206, F4/80, CD64, CD80, CD86, MHC II, CD68, CD31/PECAM-1, P-selectin, E-selectin, CD54/Intercellular Adhesion Molecule 1 (ICAM-1), CD106/vascular cell adhesion molecule 1 (VCAM-1), and/or CCR2. The specific targeting moieties can vary from tissue to tissue or organ to organ. For example, to target interstitial and alveolar macrophage in the lung, SiglecF and CD206 can be used. To target liver macrophage, CX3CR1 can be used. CD14 is a good targeting moiety for monocytes and perivascular macrophage. F4/80 can be used to generally target macrophage. CD16 can be used to target cells with FC receptors. Accordingly, in one aspect, disclosed herein are engineered nanovesicles comprising one or more macrophage targeting moieties (such as, for example an antibody specific for CD14, CD16, CX3CR1, SiglecF, CD206, F4/80, CD64, CD80, CD86, MHC H, CD68, CD31I/PECAM-1, P-selectin, E-selectin, CD54/Intercellular Adhesion Molecule 1 (ICAM-1), CD106/vascular cell adhesion molecule 1 (VCAM-1), and/or CCR2) and a CD38 inhibitor.

In one aspect, it is understood and herein contemplated that the disclosed engineered nanoparticles can be composed of any suitable material for delivery of the CD38 inhibitor to the target cell. In one aspect, the engineered nanoparticle can comprise a biocompatible polymer (such as, for example, alginate). As used herein biocompatible polymers include, but are not limited to polysaccharides such as alginate, chitosan, hyaluronic acid; hydrophilic polypeptides; proteins such as collagen, fibrin, and gelatin; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA(m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers (including triblock copolymers) thereof, and blends thereof.

In some embodiments the particle contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

1. Antibodies
(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to bind CD38. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain CD38 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bmggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The anti-CD38 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

Suitable carriers and their formulations are described in Remington: The Science and Practice of Pharmacy (19th ed.) ed. A. R. Gennaro, Mack Publishing Company, Easton, Pa. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes, exosomes, nanoparticle, or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### D. EXAMPLES

The following examples are put forth so as to provide those of ordinary skill in the art with a complete disclosure and description of how the compounds, compositions, articles, devices and/or methods claimed herein are made and evaluated; and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Utilizing ImageStream Technology to Determine Hepatatocellular CD38 Localization

CD38 is an ectoenzyme that modulates intracellular calcium levels by producing potent secondary messengers, which utilize the cellular NAD pool and lead to increased oxidative stress and inflammation. Increased CD38 expression has been associated with marginal organs, ischemia/reperfusion injury, and as a biomarker for fibrosis and transplant rejection. However, data indicate that surface expression of CD38 may not be fully predictive of function after injury or rejection, because CD38 activity and function can depend on the cell compartment distribution (plasma membrane, cytosol, nuclear membrane). Standard techniques of quantifying location have limitations. While flow cytometry is efficient, quantifying the surface and intracellular expression of the same protein can be hard to resolve. Confocal microscopy has increased resolution, but low throughput and quantification is difficult. In this study, it is demonstrated how ImageStream addresses this unmet need for a technique to determine CD38 localization.

a) Method

ImageStream is a novel technology that combines flow cytometry and microscopy. Using primary rat liver cells and novel antibody staining techniques and masking strategies, CD38 subcellular expression was quantified with high resolution, throughput, and accuracy.

b) Results

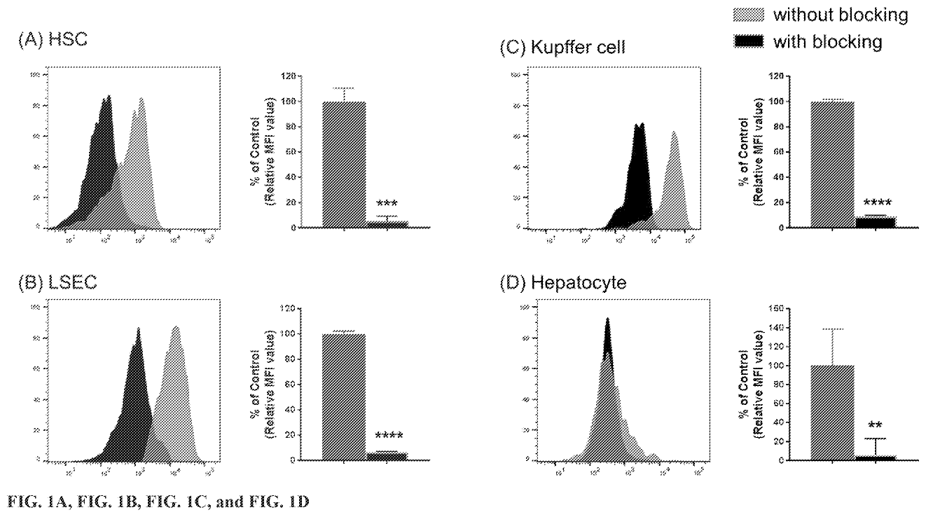

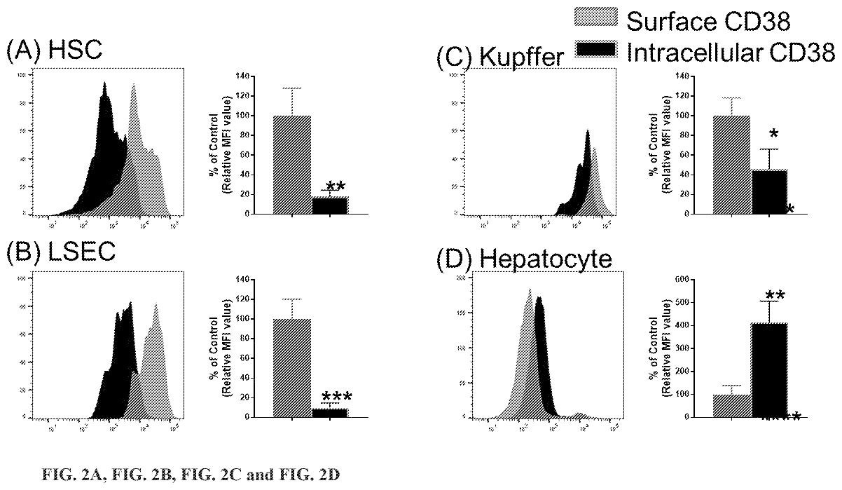

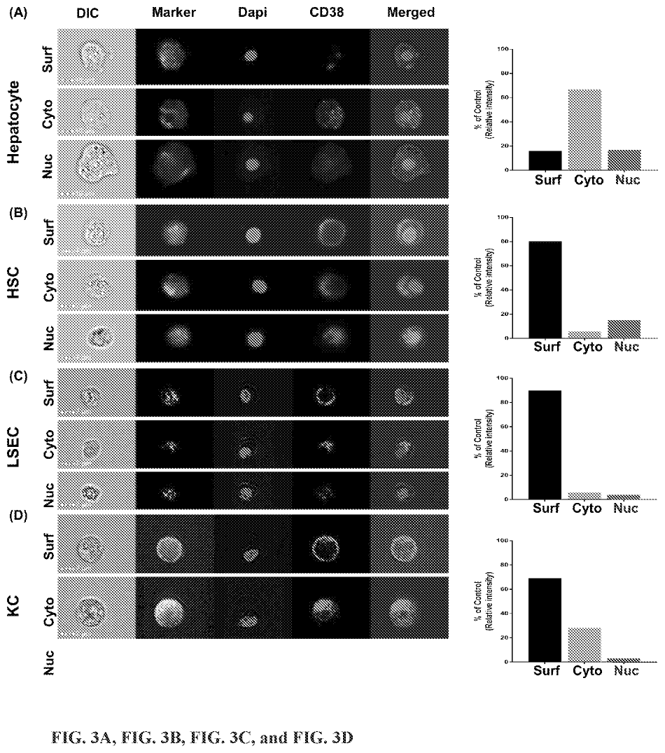

It was found that all of the four major liver cell types express CD38 to varying degrees at baseline (FIG. 1). Hepatocytes showed the lowest levels of CD38 expression with a predominance of intracellular expression, while LSECs, KCs, and HSCs show high surface expression of CD38 (FIG. 2). Moreover, we quantified CD38 cellular distribution ratio. The surface:cytoplasm:nucleus ratio for CD38 localization for cells at baseline are as follows: 16%:67%:17% for hepatocytes, 80%:6%:14% for HSCs, 90%:6%:4% for LSECs, and 69%:28%:3% for KCs (FIG. 3).
c) Discussion

Flow cytometric analysis was used along with specific surface CD38 blocking approaches to validate ImageStream masks for extracellular and intracellular CD38 expression patterns. By developing these ImageStream masking strategies, large populations of cells can be interrogated for expression and localization patterns in a manner more efficient and accurate compared to flow cytometry or confocal microscopy.

### 2. Example 2: Contribution of CD38 to Methionine Choline Deficient (MCD) Diet-Induced Liver Injury in Rats

Nonalcoholic fatty liver disease (NAFLD) and non-alcoholic steatohepatitis (NASH) are rising causes of end-stage liver disease that contribute to the increased the need for liver transplantation. As such, there is great interest in treating NASH as well as improving donor organs with NAFLD for transplantation. Herein is shown that CD38, a multifunctional ectoenzyme and modulator of intracellular calcium metabolism, may contribute to the inflammation associated with fatty livers and may serve as a therapeutic target. The role of CD38 in a methionine choline deficient (MCD) diet-induced fatty liver rat model was investigated and its contribution to inflammation and injury.

a) Method

Rat were fed a methionine choline deficient (MCD) diet to induce fatty livers and were sacrificed after 4 weeks. Blood and liver samples were obtained for measurement of ALT, AST, TG and various inflammatory cytokines. CD38 expression was determined in the liver tissue using qPCR, CD38 hydrolase and CD38 cyclase activity assays, and immunohistochemistry. CD38 expression and activity in major liver cell types was evaluated using flow cytometry and qPCR.
b) Results

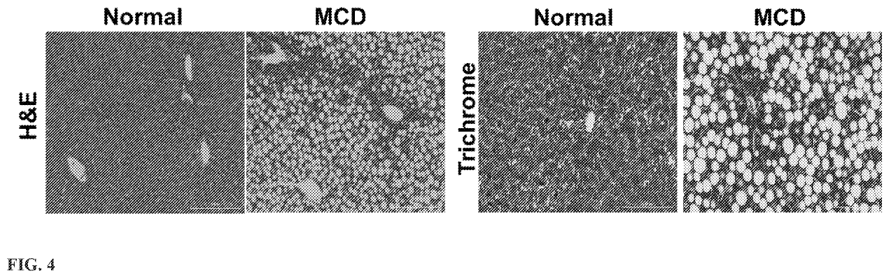

Development of fatty liver was confirmed by histology and biochemical analysis (Table 1, FIG. 1). MCD diet rats had increased ALT and AST in plasma and TG and MDA in liver tissue (Table 1) and livers showed clear steatosis (FIG. 4).

TABLE 1
Phenotypical and biochemical changes in MCD diet mice.

| | Normal Diet | MCD Diet |
|---|---|---|
| Final B.W (g) | 338.73 ± 15.55 | 202.64 ± 3.97* |
| LW/BW (%) | 3.69 ± 0.13 | 4.58 ± 0.29* |
| Plasma | | |
| ALT (mU/mL) | 44.37 ± 4.75 | 111.28 ± 23.09* |
| AST (mU/mL) | 13.27 ± 4.63 | 59.02 ± 13.69* |
| Triglyceride (mg/dl) | 106.7 ± 25.60 | 13.52 ± 8.54* |
| Liver | | |
| Liver Triglyceride (mg/g) | 7.62 ± 6.50 | 37.41 ± 10.51* |
| Liver MDA (nmol/mg) | 11.26 ± 3.20 | 55.97 ± 19.30* |

Mice fed MCD diet for 4 weeks showed changes in body weight (BW) and liver weight (LW): body weight ratio. MCD diet mice also show increased ALT, AST, and trigylcerides in plasma and increased trigylcerides and MDA in liver tissue.

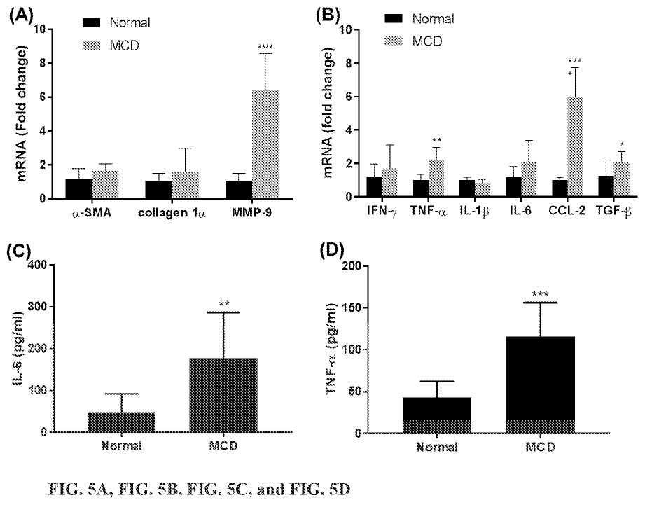

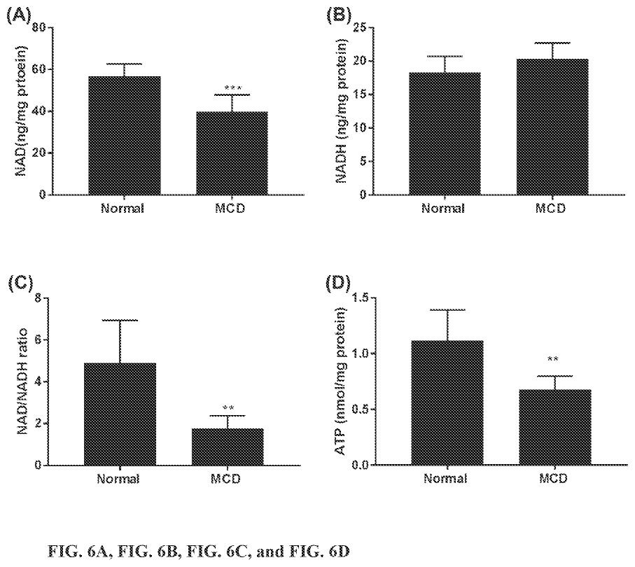

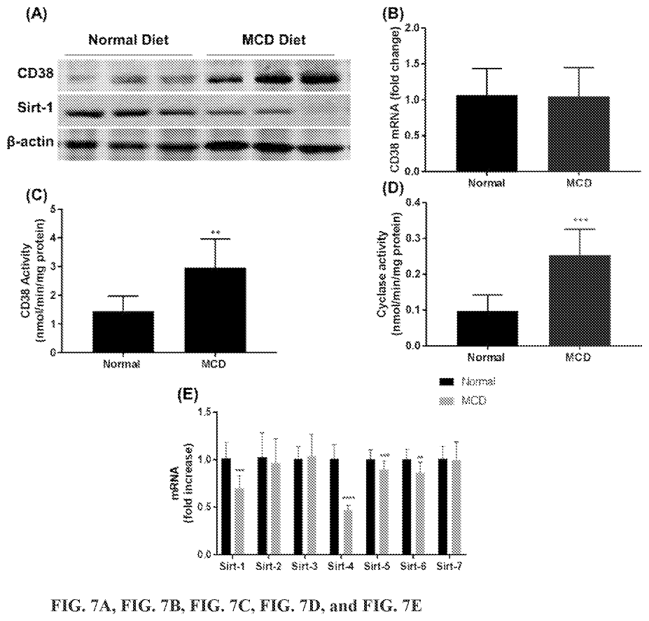

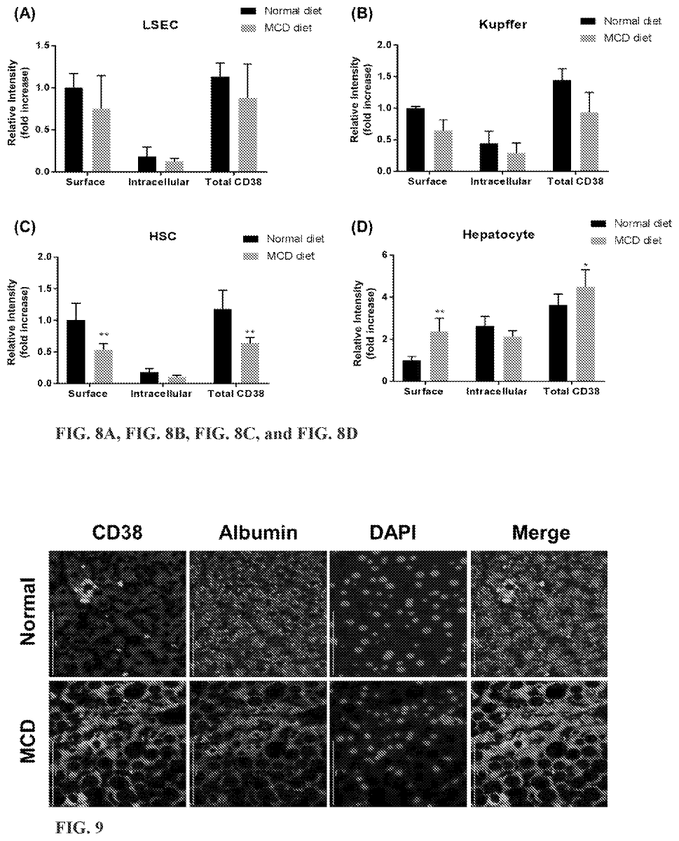

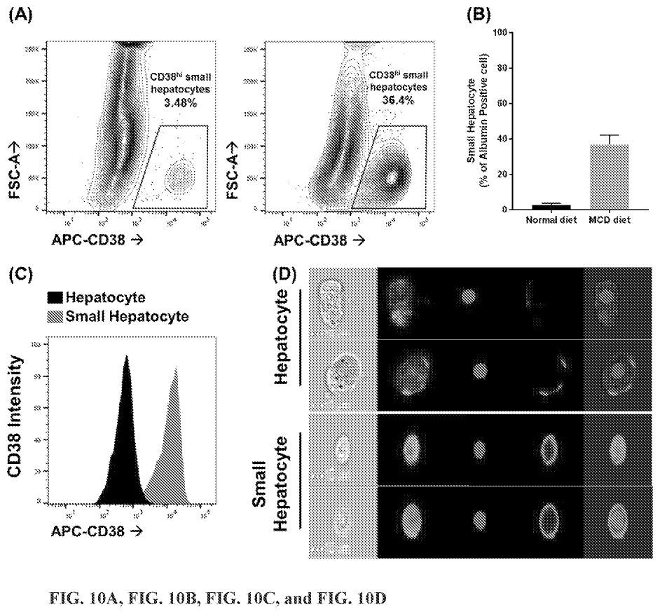

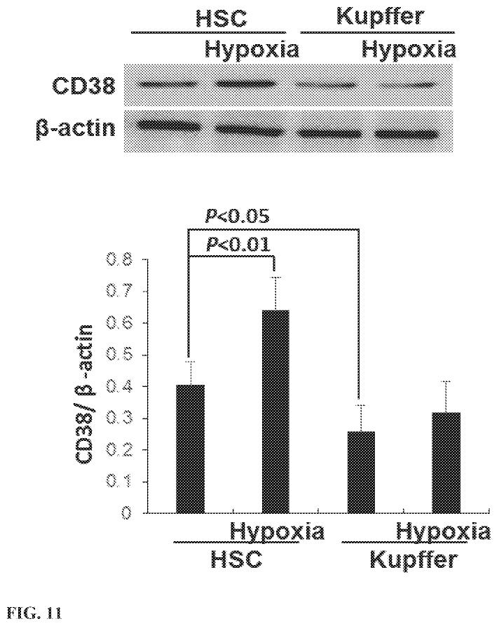

MCD diet resulted in the increased gene expression of proinflammatory cytokines including TNFα and CCL2 and markers of liver fibrosis TGFβ and MMP9 (FIG. 5). NAD, NAD/NADH ratio, and ATP were significantly reduced in the MCD diet group (FIG. 6). In hepatocytes, MCD diet decreased Sirt family gene and protein expression and increased CD38 activity and surface expression (FIG. 7) in liver tissue. This increased expression is largely driven by hepatocytes (FIGS. 8 and 9) and more specifically a population of small hepatocytes that undergo a 10-fold expansion in MCD diet livers (FIG. 10).

c) Discussion

This study demonstrates that CD38 contributes to the pathophysiology of liver injury in marginal organs via depletion of NAD pools, a common pathway in advanced age livers, ischemia/reperfusion injury, and steatosis. Additionally, a population of small hepatocytes was identified with high CD38 expression that are increased by MCD diet. These small hepatocytes drive the inflammation seen in the MCD diet livers.

### 3. Example 3: Effects of CD38 Inhibition on Hepatocellular Ischemia-Reperfusion Injury

Ischemia/Reperfusion Injury (IRI) is integral to many liver processes, including liver transplantation (LT). In LT, IRI can lead to delayed graft function (DGF) and primary graft non-function (PNF). IRI associated with the transplant process has limited the use the marginal organs for transplantation. There is great interest in mitigating IRI during LT both to improve outcomes and increase organ availability. CD38, an ecto-enzyme, drives intracellular calcium metabolism and oxidative stress. CD38 activation has been linked to organ and vascular dysfunction in some organs. In Liver CD38 activation is linked to chronic fibrosis. Prior to the present work shown herein, the role of CD38 is liver IRI remains unknown.

a) Methods

In Vitro: Primary liver cells subjected to H₂O₂-induced oxidative stress injury. In Vivo: Liver IRI induced in Sprague Dawley rats using a segmental (70% ischemia) hilar clamp model. Markers of liver injury including plasma ALT and AST, tissue MDA, ATP, and GSH, and histology assessed.
b) Results

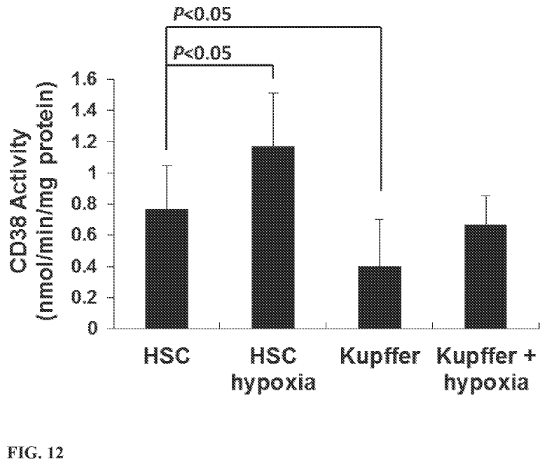

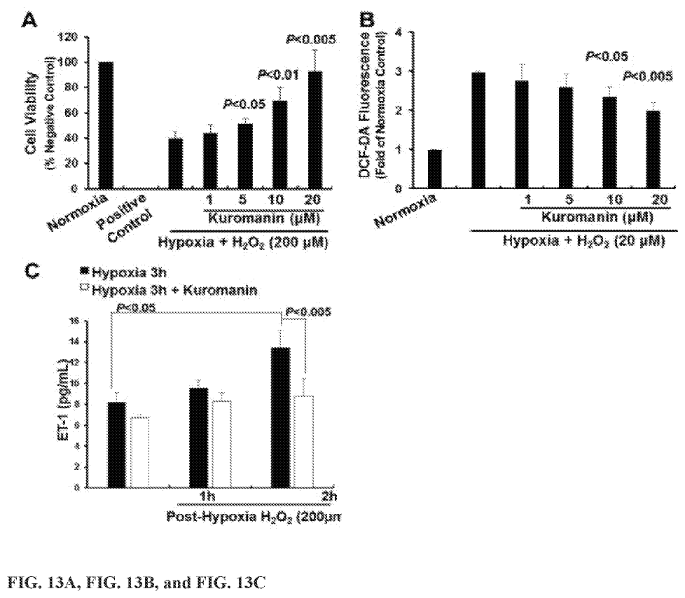

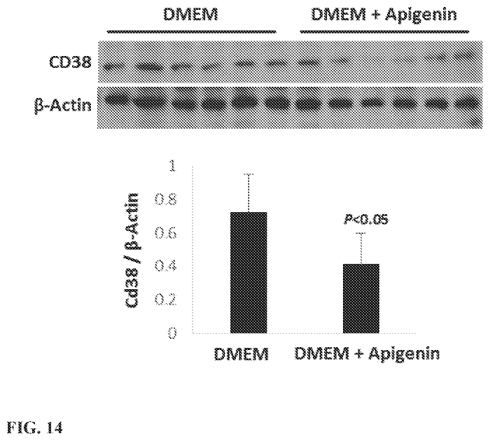

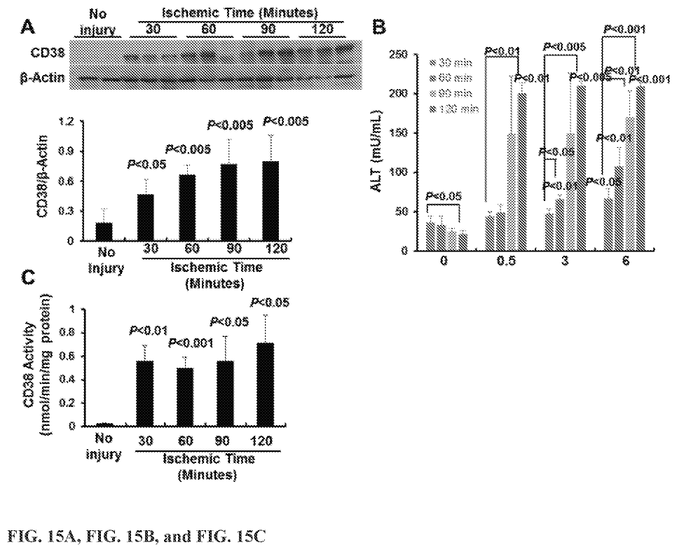

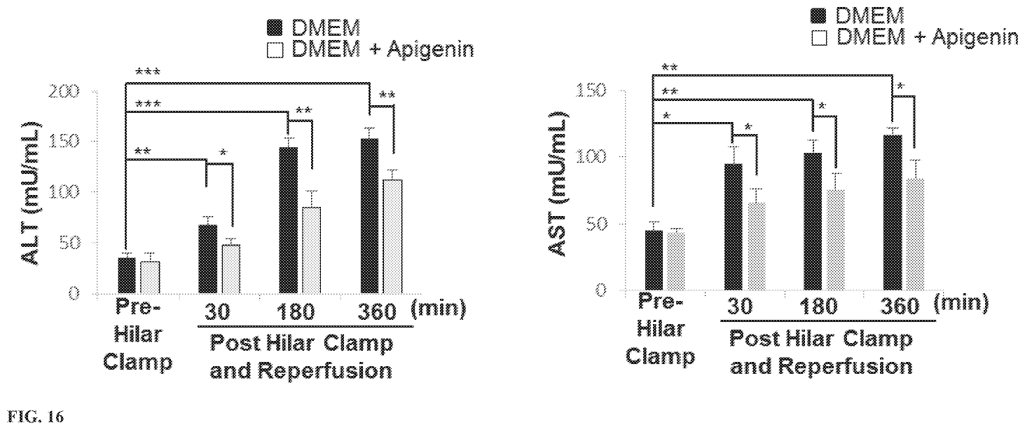

As shown in FIGS. 11 and 12, CD38 expression and activity in Kupffer cells and HSC increases during hypoxia. Kuromanin treatment resulted in improved cellular health in response to oxidative stress injury (FIGS. 13A and 13C). As shown in FIG. 14, hepatic IRI increased CD38 activity which can be rescued with Apigenin (a CD38 inhibitor). Additionally, CD38 activity correlated with increased ischemic time (FIGS. 15A and 15C). However, Apigenin treatment attenuated liver IRI (FIG. 16). It was found that Apigenin treatment improves ALT and AST. Additionally, Apigenin treatment improves tissue MAD, ATP and GSH. Lastly, Apigenin treatment preserves tissue architecture.

c) Discussion

As shown herein, CD38 plays a critical role in modulating hepatic IRI. Inhibition of CD38 provides significant protection against hepatic IRI. CDD38 activation results in downstream NAD Depletion and ROS Production. Additionally, CD38 activation results in ATP depletion and thus necroapoptosis.

### 4. Example 4: Effects of CD38 Inhibition on Inflammation and Transplantation

The approach shown herein is innovative on multiple levels: First, the total contribution of ROS to IRI in marginal (steatotic) rat donor livers after a period of warm ischemia and during normothermic ex-vivo liver perfusion (NEVLP) can be measured and defined using electron paramagnetic (EPR) spectroscopy. These organs can then be transplanted into recipient rats and the function of the organ and the degree of injury (ALT/AST) can be measured. NEVLP allows for measurement and real-time assessment of organ function and degree of injury (ALT/AST, bile output, lactate) and EPR spin probes, spin traps, and SOD mimetics allows for real-time quantification of ROS produced. Together, these platforms can measure ROS and their impact on the liver function of marginal organs pre- and post-transplantation. Second, the importance of CD38 expressed on HSC and KC (less CD38) to hepatic IRI as occurs during liver transplantation can be defined. CD38 inhibitors can be tested in vitro with HSC and KC models of hypoxia-re-oxygenation and in vivo with NEVLP and marginal vs control liver transplantation in order to determine the importance of the CD38 pathway to organ dysfunction after IRI. The findings of this work can be confirmed by using a global CD38 KO mice to perform mouse arterialized liver transplants into wild-type control mice after a period of warm ischemia. Then, a HSC-specific CD38 KO mouse can be generated to ascertain the HSC-specific CD38 contribution to IRI-mediated injury in a liver transplant model. This work can identify new therapeutic targets to mitigate IRI in marginal donor grafts. Third, the work can validate the relevance of CD38 expression/activity and HSC activation in donor livers by utilizing liver transplant patient samples and discarded donor organs. The donor organ can be biopsied prior to procurement, after cold preservation, and 2 h after implantation and restoration of blood flow. A biorepository can collect these samples and data at the time of transplant and collect clinical data at regular intervals for the rest of the life of the patient (˜100 patients per year). This provides a unique platform to study the importance of CD38, ROS, and activated HSCs in donor organs and how they relate to patient outcomes.

a) Cell Surface CD38 is Present on HSCs and a Population of KCs, but not Hepatocytes or LSECs:

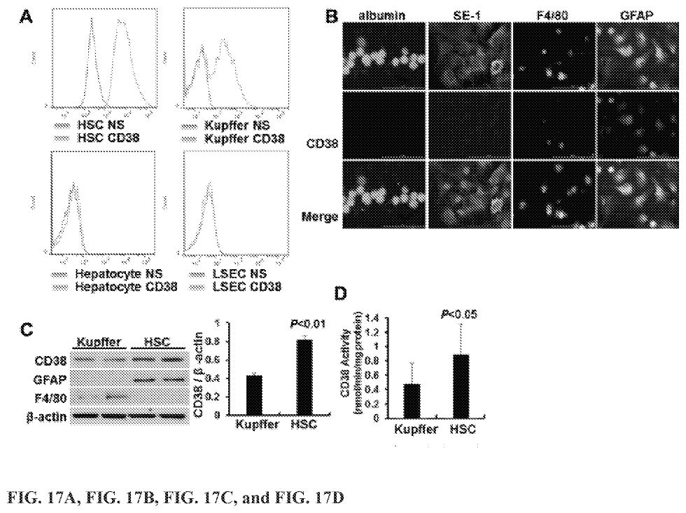

Hepatocytes, HSCs, KCs and LSECs were isolated from rat livers with high purity (96-99%; by flow cytometry). Flow cytometry and immunofluorescence show CD38 present on activated HSCs and a population of KCs, but not hepatocytes or LSECs (FIGS. 17A and 17B). Western blotting confirmed these data with HSCs expressing more CD38 than KCs (FIG. 17C). Also, CD38 enzymatic activity was greater on HSCs compared to KC (FIG. 17D). Hypoxia increased HSC CD38 expression and activity: Primary rat HSCs and KCs were exposed to 6 h hypoxia.

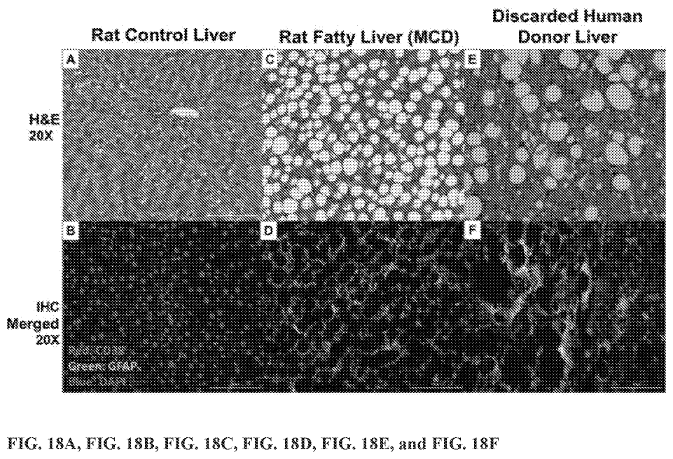

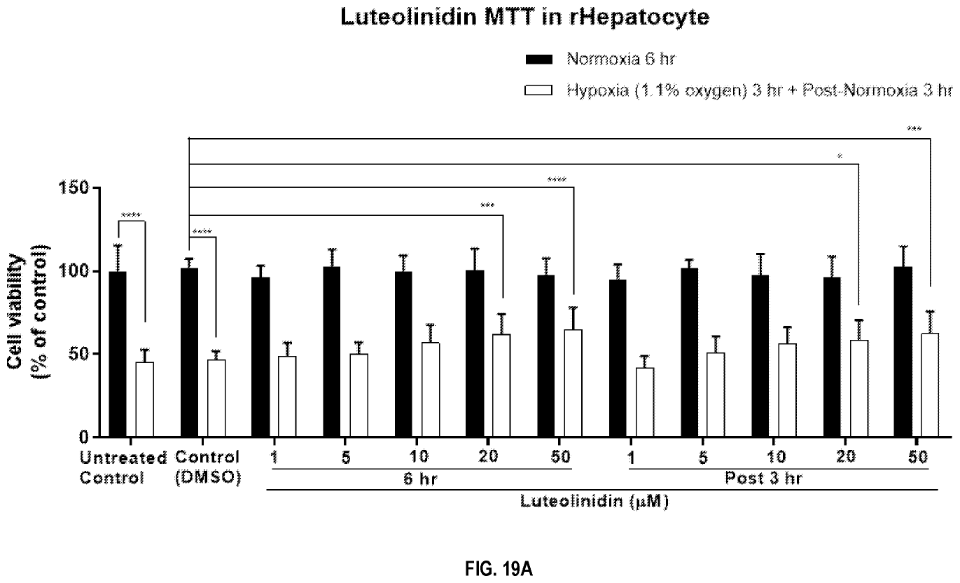

Hypoxic HSCs had increased CD38 expression (FIG. 11) and CD38 activity measured by fluorometric assay (FIG. 12). CD38 expression, activity, and hepatocellular injury increased with duration of ischemia in a rat hilar clamp model of IRI: Sprague-Dawley rats underwent a hilar clamp procedure to induce IRI for 30, 60, 90, 120 min of ischemia followed by 6 h reperfusion. Tissue CD38 expression was measured by Western blot (FIG. 15A), serum ALT by colorimetric assay at 0, 30, 180, 360 min post reperfusion (FIG. 15B), and CD38 activity at all ischemia time points (FIG. 15C). CD38 inhibition increases cell viability, decreases intracellular ROS formation and ET-1 production in HSCs exposed to hypoxia/oxidant stress: Primary rat HSCs were exposed to 3 h hypoxia (0% 02) and 2 h oxidant stress (H₂O₂). Cells treated with the CD38 inhibitor Kuromanin demonstrated increased viability (FIG. 13A), decreased ROS production (FIG. 13B), and decreased ET-1 release (FIG. 13C). Apigenin reduces hepatic IRI: Bioflavonoid CD38 inhibitor Apigenin delivered intra-hepatically with a hilar clamp model (FIG. 6A) demonstrated protection from injury compared to controls after 1 h of warm ischemia followed by a 6 h of reperfusion (FIG. 6B). Rat model of marginal donor organ recapitulates the inflammatory environment of a discarded “high risk” human donor organ: Control rats have little steatosis by H&E (FIG. 18A) and mild CD38 expression by IHC (FIG. 18B). Rats fed a methionine choline deficient (MCD) diet have severe steatosis by H&E (FIG. 18C) and high CD38 expression co-localizing with GFAP positive HSCs by IHC (FIG. 18D). Discarded human donor liver shows severe steatosis by H&E (FIG. 18E) and moderate CD38 expression co-localizing with HSCs by IHC (FIG. 18F).

b) The Importance of ROS Production to IRI in Marginal Livers.

ROS are key mediators of IRI during liver transplantation, but it is not yet defined how ROS relates to exacerbated injury and organ dysfunction with a marginal liver post-transplantation. EPR spectroscopy during NEVLP with subsequent transplantation allows for the quantification of ROS production in marginal organs. These results can be correlated with liver injury and measurements of inflammatory stress. Marginal livers (ML) can be generated in rats fed a methionine choline-deficient (MCD) diet, measure ROS production, hepatic injury and inflammatory stress and compare this with perfused control livers (CL). Then, the ML and CL can be transplanted to control rats in order to quantify relative graft injury, inflammation, survival. ROS production along with immunohistochemistry (IHC) post transplantation can be performed to delineate cell specific contribution to IRI. In one aspect, these studies tests the hypothesis that ROS can be measured during NEVLP and that there is a difference between ROS production in MLs compared to CLs. Additionally, the studies quantify graft injury and recipient survival after transplantation of MLs and CLs into control rats and also determines cell-specific ROS contribution to IRI post-transplantation using IHC.

(1) Normothermic Ex Vivo Liver Perfusion:

Marginal livers (high steatosis and inflammation) can be generated in Sprague-Dawley rats fed a MCD diet. Rats can be euthanized at 1, 2, 3, and 4 weeks, and the degree of inflammation and large droplet steatosis can be determined by liver pathologist, who can histologically grade blinded biopsies into none: control, mild: <30%, moderate: 30-60%, and severe: >60%. These categories are analogous to clinical parameters, correlative to length of time on MCD, and can be used to determine groupings for post-experiment analysis. After 15-30 min ischemia by hilar clamp, donor livers can undergo NEVLP for up to 4 h. ROS can be measured in the pre- and post-organ perfusate using EPR spectroscopy with spin traps (such as BMPO [5-tert-Butoxycarbonyl-5-methyl-1-pyrroline-N-oxide]) or spin probes (such as CMH). Hepatic transaminases (ALT/AST), electrolytes (K, Na, Cl), glucose, pH, and O₂ consumption, and inflammatory cytokine levels (IL-1, IL6, TNFa, ET-1-by ELISA) can be measured in the perfusate. Appropriate control rat donors fed a normal diet can be used as a control. Samples can be assayed and analyzed in triplicate and compared to appropriate controls.

(2) Liver Transplants.

After NEVLP, livers can be transplanted into recipient rats and serum levels of transaminases followed for 3 days. At day 3, before liver procurement, ROS measurements can be performed using EPR spin trap or probe infused in the portal vein and collected from inferior vena cava. ALT/AST and cytokines can also be measured in the serum. Rats can be euthanized, and livers collected for histology (H&E, Tunnel), IHC, Western blot, and qPCR. Tissue samples can be interrogated for CD38 expression (Western) and activity (fluorometric assay), inflammatory cytokines expression (qPCR and ELISA), and HSC quiescence by IHC (desmin, LRAT, GFAP) vs. activation (PDGFR, aSMA, collagen I, and CTGF).

(3) Measurement of Cell-Specific ROS Contribution to IRI Post-Liver Transplantation.

Liver tissue can be cryo-embedded in OCT and cut into 8-μm transverse sections. Superoxide generation in liver tissue can be measured by covering sections with a probe solution of the redox dye dihydroethidium (DHE; 10 μM) along with the nuclear stain DRAQ5 in absence or presence of the SOD mimetic MnTBAP (50 μM) to confirm specificity for superoxide. Co-localization can be performed using the antibody appropriate for the following cell types: hepatocytes (albumin), LSEC (SE-1), KC (F40/80) and quiescent HSC can beidentified by the presence of desmin, LRAT, or GFAP or activated HSC by their enhanced expression of PDGFR, aSMA, collagen I, and CTGF in sequential sections. Samples can be assayed and analyzed in triplicate and compared to appropriate controls and freshly procured ML and CL.

(4) Results

PDGFR, aSMA, collagen, and CTGF expression increase for HSCs that are more of an activated rather than quiescent state. Increasing activation of HSCs can be observed as degree of steatosis increases, which is consistent with the data shown herein (FIG. 18). ROS are critical mediators of the cellular dysfunction seen with IRI, and increased radical signal in the perfusate of the livers that are highly steatotic is observed. Increased hepatic transaminases and hepatocyte necroptosis (histology) associated with the increased ROS can also be observed. Elevated ROS detection occurs before any rise in transaminases or inflammatory cytokine production. The amount of donor organ dysfunction with liver transplantation in the rat model correlates with degree of steatosis, ROS production and CD38 expression/activity. Additionally, increased ROS production and inflammatory cytokine release in the activated HSCs is observed when compared with non-activated HSC exposed to an IRI.

c) Delineate the Role of the HSC During IRI by Defining the Importance of CD38 as an Inflammatory Mediator.

The role of HSCs in acute inflammation and IRI during liver transplantation is not well-defined, but recent data indicates that HSCs are an important mediator of injury. The role of CD38 in inflammation and protection from IRI is well-described in other organs like heart and brain, but not the liver. In liver, HSCs have the highest expression of CD38 (some KCs also express CD38) and are potent producers of pro-inflammatory mediators and ROS during acute injury, but HSCs have not been well-studied in this context. Herein is shown importance of CD38 in hepatic IRI as occurs with transplantation, and by using a mouse model of IRI with KO technology. These studies focus on the role and importance of CD38 as a key mediator of the HSC inflammatory response and enhancement of IRI. Studies in the rat can then be confirmed using global CD38 KO mice both in vitro (HSC and KC IRI) and in vivo for liver transplantation into control mice. HSC-specific CD38 KO can delineate the role of HSC mediated by CD38 in IRI. Herein, CD38-specific inhibitors are tested with in vitro hypoxia/reoxygenation-oxidant stress using rat HSCs and KCs. The effects of CD38 inhibition (CD38i) compared to vehicle control (VC) in ML and CL is tested with measurements of ROS (EPR), injury, and inflammatory stress during NEVLP. Next, the effects of CD38i vs. VC in ML and CL post-transplantation to control rats is tested. The role of CD38 in relative graft injury, inflammation, and ROS, as well as longitudinal outcomes can be determined. Then, HSC and KC are isolated from global CD38 KO and WT mice and measure ROS, inflammation, and activation markers during in vitro IRI.

(1) CD38 Inhibition in HSCs and KCs and IRI.

HSCs and KCs isolated from rats can be exposed to hypoxia (0% O₂) and/or oxidant stress (H₂O₂) in the presence or absence of a dose response curve of inhibitors (e.g., Luteolindin). CD38 activity, cell viability (MTT), ROS (EPR) and NOX expression (Western blot), intracellular calcium release and cADPR, NAADP expression (Western blot), and iNOS expression (Western blot). Appropriate vehicle, non-treated, and single variable controls can be used. Samples can be assayed and analyzed in triplicate and compared to appropriate controls.

(2) CD38 Inhibition in ML and CL During NEVLP.

Using the most effective CD38 inhibitor and concentration determined herein, ML and CL with inhibitor vs. a VC is compared during NEVLP.

(3) Liver Transplantation of ML and CL into Normal Rat Recipients.

Livers are transplanted into normal rat recipients using the methodology and measurements described herein. Appropriate vehicle, non-treated, and single variable controls can be used. Samples can be assayed and analyzed in triplicate and compared to appropriate controls.

(4) Isolation and IRI in Global CD38KO Mouse HSC and KC.

Mouse HSC and KC can be isolated from global CD38 KO and wild-type (WT) control mice, and expose them to hypoxia (0% O₂) and oxidative stress (H₂O₂). ROS production can be evaluated with 2′,7′ dichlorofluorescin diacetate (DCFDA), DHE, and also EPR spin trapping. Cell viability (MTT), pro-inflammatory cytokine release (IL-1, IL-6, ET-1, and TNFa; ELISA), and, as described herein, HSC activation markers can be measured (IHC, qPCR, and Western blot). Appropriate non-treated, WT, and single variable controls can be used. Samples can be assayed and analyzed in triplicate and compared to appropriate controls.

(5) Liver Transplant Using CD38 KO Mice:

A period of 15-30 min warm ischemia time can be performed on CD38 KO or HSC-specific CD38 KO donor livers prior to transplant. Mouse arterialized liver transplant can be performed with KO donor livers and WT recipient mice. Mice can be followed, euthanized at 3 days, and livers procured. Daily serum ALT measurements can be taken to quantify the magnitude of hepatic injury. H&E, Tunnel, Western blot, IHC, and qPCR to quantify inflammatory cytokine expression, necroptosis, and HSC activation (as in rat experiments) can be performed on the liver tissue. WT donor to WT recipients can serve as a control. For HSC-specific CD38 KO mice, Pdgfrb-Cre-only littermates can be used as controls. Samples can be assayed and analyzed in triplicate and compared to appropriate controls.

(6) Results:

Global CD38 KO or CD38 HSC-specific KO and WT or Pdgfrb cre-only mice can be used with liver transplant models to confirm findings with CD38 inhibitors in rats. The IRI in the liver transplant and in vitro cell models of CD38 inhibition/KO can be reduced, with the CD38-deficient cells and animals exhibiting less inflammation and injury compared to the controls. PDGFR, aSMA, collagen, and CTGF can increase in HSCs that are more inflamed compared to CD38 inhibited/KO HSCs. Inflammatory cytokines and ROS in the perfusate (rats) and in the tissue (rats and mice) also decrease with CD38 inhibition or KO.

d) Determine the Role of HSCs and CD38 in Allograft Injury and Function in Liver Transplant Patients.

Marginal donors are more susceptible to IRI and organ dysfunction after transplantation. HSCs in marginal donor organs are skewed toward an activated or inflammatory phenotype and can account for much of the enhanced injury after transplantation. Herein the markers of HSC activation and inflammation are determined. CD38 expression/activity is also measured from biopsies of donor livers prior to procurement, after cold preservation, and 2 h after implantation and reperfusion. Next, graft function and recipient health from transplantation to 36 months post-transplantation is analyzed. Lastly, values of inflammation, oxidative radicals, CD38, and functionality are measured in cells and tissue of discarded livers not suitable for transplantation.

(1) Liver Transplant Patients

Herein is provided operationally defined and modeled marginal donor organs as having moderate to high degrees of macrovesicular steatosis and varying inflammation, thus reflecting the most common type of marginal donor in the U.S. Oxidative stress and HSC activation are thought to be important in the pathogenesis of NAFLD and can explain the enhanced injury seen with the use of these organs. Donor livers clinically classified as marginal in the program meet one or more of the characteristics outlined in Table 2 and may make up ˜15 to 30% of the donor organs transplanted. Herein samples from all of the consented liver transplant patients (80-100 liver transplants per year) as well as discarded organs from an organ procurement organization, Lifeline of Ohio (˜10-15 per year) are investigated. Potential recipients for liver transplantation can be identified, consented, and samples and data procured by the biorepository. The biopsies recovered can be wedge and core needle biopsies from both the right and left hepatic lobes to minimize regional variability of inflammatory markers. They can be taken at the time of donor procurement prior to cross clamp and preservation, when the liver is removed from the cold preservation solution, and final biopsies can be taken 1.5-2 h after reperfusion prior to abdominal incision closure. Tissue samples can be immediately placed in formalin, snap frozen, or placed in AllProtect (Qiagen) to allow for a variety of downstream processing. Clinical data and patient outcomes from the medical records are recorded by a biorepository and can be distributed as coded data.

TABLE 2

| High Risk Organ | Measurements | Measurements | |
|---|---|---|---|
| (Marginal) | (Tissue) | (Serum) | Outcome Metrics |
| Donation after | CD38 Expression | Markers of Liver | Graft Loss |
| Cardiac Death (DCD) | (Western) and | Injury/Function | |
| | Activity | (AST, ALT, ALP, | |
| | | Bilirubin) | |
| Elevated | Markers Hepatic | Lactate Clearance | Patient Survival |
| Transaminases (AST | Stellate Activation by | | |
| or ALT >500) | IHC (desmin, LRAT, | | |
| | GFAP, PDGFR, αSMA, | | |
| | collagen a1(I), CTGF) | | |
| Macrosteatosis | ET-1 (RT-PCR) | Coagulation | Acute Rejection |
| of >30% | | Perimeters | Episodes |
| | | (PT/PTT/INR) | |
| Age >65 | iNOS, eNOS, NOX | | Chronic Rejection |
| | expression (Western) | | |
| HCV positive donor | H&E and TUNNEL | | Graft function |
| | Staining (histologic | | during index |
| | grading by pathologist | | hospitalization |
| | of steatosis and injury) | | |
| Donor Risk Index | ROS (EPR Spectroscopy | | Graft Function at |
| (DRI) >1.9 | of tissue) | | months 1, 3, 6, |
| | | | 12, 24 and 36 |

(2) Discarded Organs

Donor organs not suitable for transplantation can be obtained by the biorepository prior to donor cross-clamp and preservation. Samples can be harvested as above with the addition of fresh tissue placed in ice cold preservation media (MACs Tissue Storage Solution). For both patient samples and discarded organs, the formalin and AllProtect samples can be processed for markers of HSC activation, inflammation, hepatocellular injury, and oxidative stress (Table 2). The snap frozen samples can be processed for ROS quantification and CD38 activity. Clinical data (Table 2) and patient outcomes (Table 2) can be compared for at least 36 months. In vitro experiments with discarded organ KCs and HSCs can have similar measurements to others described herein. Discarded organs can serve as positive controls for comparison to the transplanted marginal organ biopsies. Additionally, samples and clinical data from standard criteria organs can be used in this study and can serve as a control for the different types of marginal organs. Finally, the pre-transplant donor samples can serve as baseline measurements of organ quality. Appropriate single variable and untreated controls can be used. Samples can be assayed and analyzed in triplicate and compared to appropriate controls.

(3) Results:

The findings shown herein correlate with animal and in vitro models of IRI. Marginal donors (depending on the background variables e.g., DCD vs steatosis) can exhibit more allograft dysfunction, increased ROS/oxidant stress, and CD38 activation. HSCs from marginal organs exhibit greater activation/inflammation and more allograft dysfunction than standard organs. The most interesting comparison can be between discarded organs and marginal organs used for transplantation. Data from these groups and molecular comparisons are important in evaluating the use of these organs and the acceptance criteria.

### Example 5: Hypoxia Reoxygenation In Vitro Studies with Luteolinidin and 78C

a) Methods

Rat hepatocytes were cultured overnight in hypoxia conditions (1.1% O₂) in the presence of 1-50 μM 78C or vehicle only in glucose free media for 3 hours. After the three hour incubation in hypoxic conditions, cells were incubated an additional 3 hours in normoxic conditions. Cell viability was determined by MTT ((4,5-dimethylthiazol-2yl)-2,5-diphenyl-2H-tetrazolium bromide)) stain

b) Results

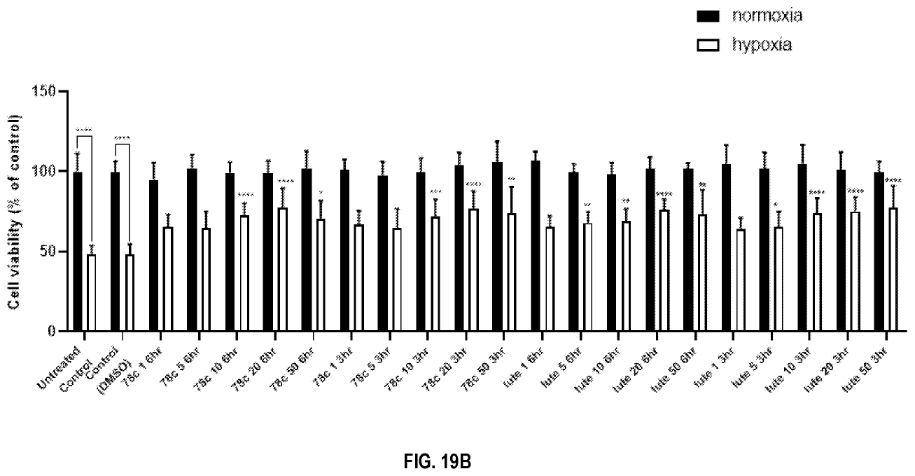

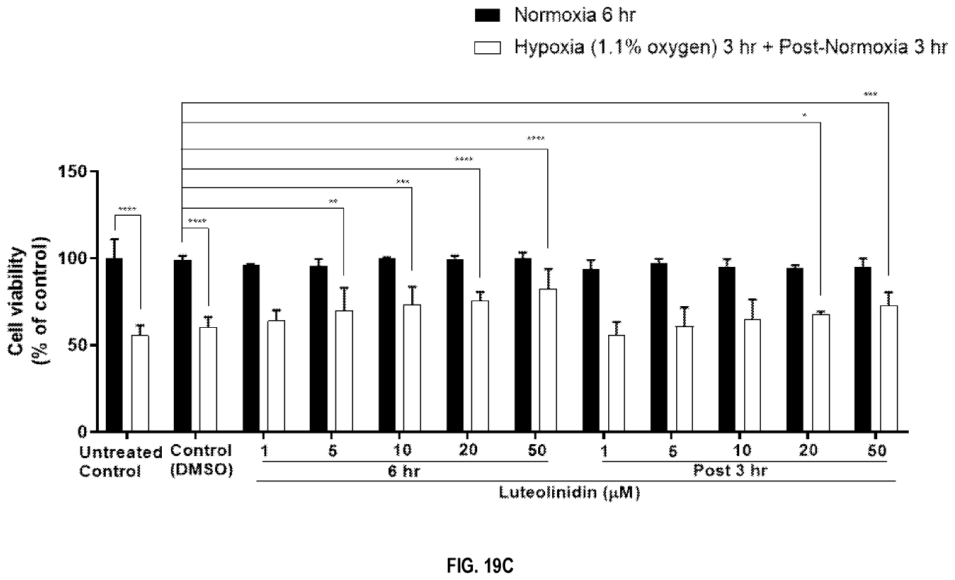

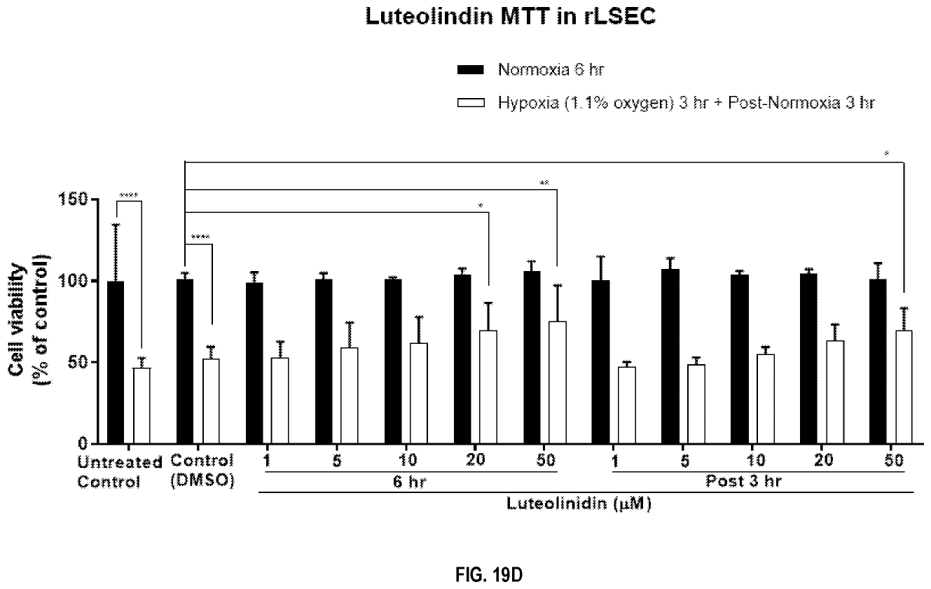

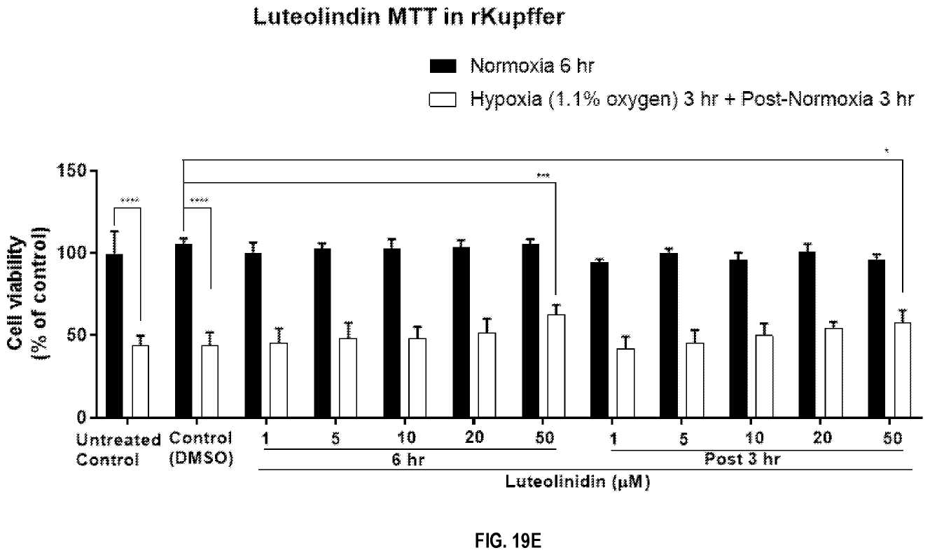

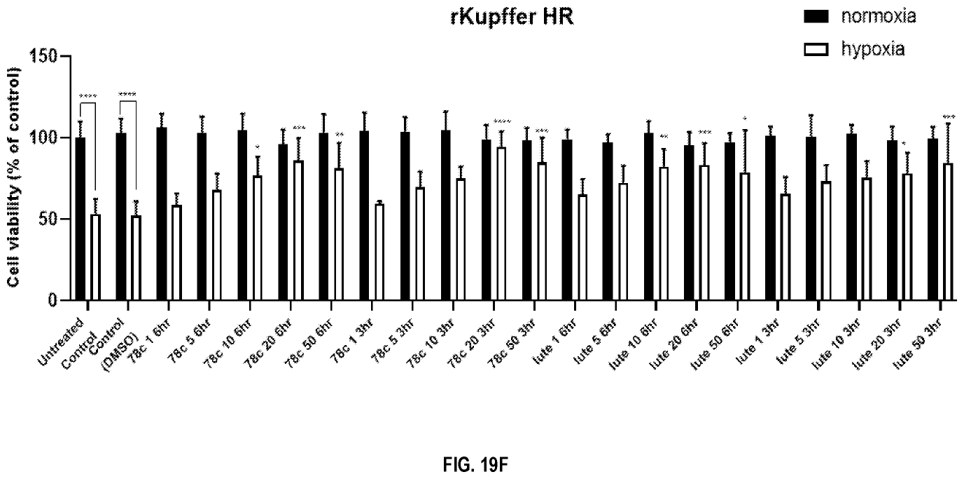

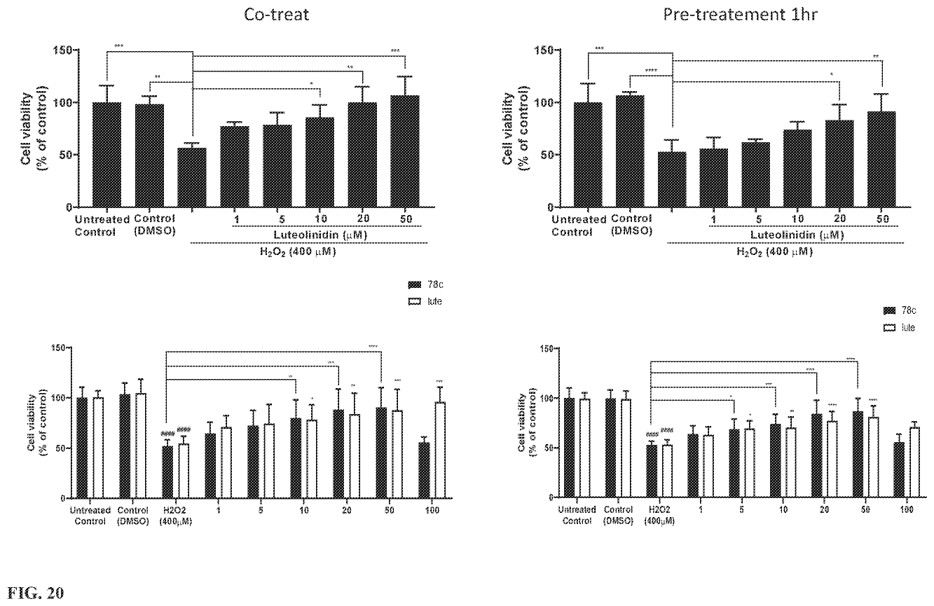

As shown in FIG. 19 cell viability increased over nontreated controls in both the 78C and luteolinidin treated rat hepatocytes (19A and 19B), rat hepatic stellate cells (19C), rat sinusoidal endothelial cells (19D), or Kupffer cells (19E and 19F). Pretreatment and co-treatment also increased viability of cells over controls (FIG. 20).

### E. REFERENCES

Bennett K M, Scarborough J E, Pappas T N, Kepler T B. Patient Socioeconomic Status Is an Independent Predictor of Operative Mortality. Annals of Surgery. 2010; 252.
Haider A H, Scott V K, Rehman K A, et al. Racial Disparities in Surgical Care and Outcomes in the United States: A Comprehensive Review of Patient, Provider, and Systemic Factors. Journal of the American College of Surgeons. 2013; 216:482-492.e412.
Quillin R C, III, Wilson G C, Wima K, et al. Neighborhood Level Effects of Socioeconomic Status on Liver Transplant Selection and Recipient Survival. Clinical Gastroenterology and Hepatology. 2014; 12:1934-1941.
Risk Adjustment for Socioeconomic Status or Other Sociodemographic Factors. Risk Adjustment and SES: National Quality Forum; 2014.
Ross K, Patzer R E, Goldberg D S, Lynch R J. Sociodemographic Determinants of Waitlist and Posttransplant Survival Among End-Stage Liver Disease Patients. American Journal of Transplantation. 2017.
Schold J D, Phelan M P, Buccini L D. Utility of Ecological Risk Factors for Evaluation of Transplant Center Performance. American Journal of Transplantation. 2017; 17:617-621.
Yoo H Y, Thuluvath P J. Outcome of liver transplantation in adult recipients: Influence of neighborhood income, education, and insurance. 2004; 10:235-243.

1. An engineered nanovesicle comprising a targeting moiety and an inhibitor of CD38; wherein the targeting moiety targets endothelium or macrophage contained in interstitial compartments of a tissue or organ of interest.

2. The engineered nanovesicle of claim 1, wherein the inhibitor of CD38 is a RNAi, oligonucleotide, antibody, or small molecule.

3. The engineered nanovesicle of claim 2, wherein the inhibitor of CD38 comprises a thiazoloquin(az)olin(on)e compound, apigenin, kuromanin, or luteolinidin.

4. The engineered nanovesicle of claim 3, wherein the thiazoloquin(az)olin(on)e compound comprises 78C.

5. The engineered nanovesicle of claim 1, wherein the targeting moiety comprises a macrophage targeting moiety and wherein the macrophage targeting moiety comprises an antibody that targets CD14, CD16, CX3CR1, SiglecF, CD206, F4/80, CD64, CD80, CD86, MHC II, CD68, CD31/PECAM-1, P-selectin, E-selectin, CD54/Intercellular Adhesion Molecule 1 (ICAM-1), CD106/vascular cell adhesion molecule 1 (VCAM-1), and/or CCR2.

6. A pharmaceutical composition comprising the engineered nanovesicle of claim 1.

7. A method of treating an inflammatory disease comprising administering to a subject with an inflammatory liver disease the engineered nanovesicle of claim 1.

8. The method of treating an inflammatory disease of claim 7, wherein the inflammatory disease comprises an inflammatory liver disease.

9. The method of treating an inflammatory disease of claim 8, wherein the inflammatory liver disease comprises Hyperlipidemia, fatty liver disease (steatosis), steatohepatitis, metabolic syndrome, Phenylketonuria (PKU), Maple syrup urine disease (MSUD), Gaucher's disease, hypercholesterolemia, hypertriglyceridemia, hyperthyroidism, hypothyroidism, dyslipidemia, hypolipidemia, and galactosemia, Alcoholic liver disease (ALD), or non-alcoholic fatty liver disease.

10. The method of treating an inflammatory disease of claim 7, wherein the inflammatory disease comprises an inflammatory lung disease.

11. The method of treating an inflammatory lung disease of claim 10, wherein the inflammatory lung disease comprises acute lung injury, acute respiratory distress syndrome (ARDS), transfusion induced acute lung injury (TRALI), or ventilator induced lung injury.

12. A method of preparing a donor organ or tissue for transplant comprising contacting the organ or tissue with the engineered nanovesicle of claim 1.

13. The method of preparing a donor organ or tissue for transplant of claim 12, wherein the donor or tissue comprises liver, lung, heart, kidney, trachea, pancreas, bones, skin, tendons, cornea, vascular tissue, or heart valves.

14. The method of preparing an organ or tissue for transplant of claim 12, wherein the engineered nanovesicle is delivered to a donor subject comprising the donor tissue or organ prior to removal of the organ or tissue.

15. The method of preparing an organ or tissue for transplant of claim 12, wherein the engineered nanovesicle is delivered to the organ or tissue via ex vivo organ perfusion, solution flush, cold static storage solution, or normothermic solution.

16. A method of inhibiting, reducing, or repairing tissue damage to a donor organ or tissue during a transplantation procedure comprising contacting the organ or tissue with the engineered nanovesicle of claim 1.

17. The method of inhibiting, reducing, or repairing tissue damage of claim 16, wherein the donor or tissue comprises liver, lung, heart, kidney, trachea, pancreas, bones, skin, tendons, cornea, vascular tissue, or heart valves.

18. The method of inhibiting, reducing, or repairing tissue damage of claim 16, wherein the engineered nanovesicle is delivered to a donor subject comprising the donor tissue or organ prior to removal of the organ or tissue.

19. The method of inhibiting, reducing, or repairing tissue damage of claim 16, wherein the engineered nanovesicle is delivered to the organ or tissue via ex vivo organ perfusion, solution flush, cold static storage solution, or normothermic solution.

20. The method of inhibiting, reducing, or repairing tissue damage of claim 16, wherein the engineered nanovesicle is delivered to the organ or tissue before and/or after transplantation.

21. A method of treating an inflammatory disease comprising administering to a subject with an inflammatory disease an inhibitor of CD38.

22. The method of treating an inflammatory disease of claim 21, wherein the CD38 inhibitor is delivered via an engineered nanovesicle comprising a targeting moiety that targets endothelium or macrophage contained in interstitial compartments of a tissue or organ of interest.

23. The method of treating an inflammatory disease of claim 22, wherein the macrophage targeting moiety comprises an antibody that targets CD14, CD16, CX3CR1, SiglecF, CD206, F4/80, CD64, CD80, CD86, MHC II, CD68, CD31/PECAM-1, P-selectin, E-selectin, CD54/Intercellular Adhesion Molecule 1 (ICAM-1), CD106/vascular cell adhesion molecule 1 (VCAM-1), and/or CCR2.

24. The method of treating an inflammatory disease of claim 21, wherein the wherein the inhibitor of CD38 is a RNAi, oligonucleotide, antibody, or small molecule.

25. The method of treating an inflammatory disease of claim 24, wherein the inhibitor of CD38 comprises a thiazoloquin(az)olin(on)e compound, apigenin, kuromanin, or luteolinidin.

26. The method of treating an inflammatory disease of claim 25, wherein the thiazoloquin(az)olin(on)e compound comprises 78C.

27. The method of treating an inflammatory disease of claim 21, wherein the inflammatory disease comprises an inflammatory liver disease.

28. The method of claim 27, wherein the inflammatory liver disease comprises Hyperlipidemia, fatty liver disease (steatosis), steatohepatitis, metabolic syndrome, Phenylketonuria (PKU), Maple syrup urine disease (MSUD), Gaucher's disease, hypercholesterolemia, hypertriglyceridemia, hyperthyroidism, hypothyroidism, dyslipidemia, hypolipidemia, and galactosemia, Alcoholic liver disease (ALD), or non-alcoholic fatty liver disease.

29. The method treating an inflammatory liver disease of claim 28, wherein the non-alcoholic fatty liver disease comprises non-alcoholic fatty liver (NAFL) or non-alcoholic steatohepatitis (NASH).

30. The method of treating an inflammatory disease of claim 21, wherein the inflammatory disease comprises an inflammatory lung disease.

31. The method of treating an inflammatory lung disease of claim 30, wherein the inflammatory lung disease comprises acute lung injury, acute respiratory distress syndrome (ARDS), transfusion induced acute lung injury (TRALI), or ventilator induced lung injury.
